(19) United States
(12) Patent Application Publication
(10) Pub. No.: US 2011/0207168 A1
(43) Pub. Date: Aug. 25, 2011

(54) Fiber-Based Biosensors for Use in Detecting the Presence of a Biologically Active Substance

(76) Inventors: Konstantin G. Kornev, Clemson, SC (US); Aleksey Vertegel, Easly, SC (US); Igor Luzinov, Central, SC (US)

(21) Appl. No.: 13/061,134

(22) PCT Filed: Aug. 26, 2009
(86) PCT No.: PCT/US2009/004848

§ 371 Date: Apr. 6, 2011

Related U.S. Application Data
(60) Provisional application No. 61/092,180, filed on Aug. 27, 2008.

(51) Int. Cl. C12Q 1/04

(52) U.S. Cl. 435/34

(57) ABSTRACT

Disclosed are fibers comprising one or more electrostatically attached substrates that can be used to determine the presence of a biologically active substance. Further disclosed are substrates comprising the fibers, articles of manufacture comprising the fibers and/or substrates, and methods for detecting the presence of a biologically active substance.

### CROSS-REFERENCE TO RELATED APPLICATIONS

This application claims benefit of priority to U.S. Provisional Application Ser. No. 61/092,180 filed Aug. 27, 2008, which is herein incorporated by reference in its entirety.

### FIELD

Disclosed are fibers comprising one or more electrostatically attached substrates that can be used to determine the presence of a biologically active substance. Further disclosed are composites comprising the fibers, articles of manufacture comprising the fibers and/or substrates, and methods for detecting the presence of a biologically active substance.

### BACKGROUND

Bacterial vaginosis (BV) is a common medical syndrome affecting millions of women annually. The syndrome is the most common vaginal condition among women of childbearing age and is associated with approximately one-third of all cases of vulvovaginitis. BV is commonly associated with preterm delivery, prematurity, pelvic inflammatory disease, and the acquisition of the human immunodeficiency virus (HIV). Research has also identified BV as a risk factor for histologic chorioamnionitis, amniotic fluid infection, post cesarean endometritis, and various other pregnancy complications.

Bacterial vaginosis is defined microbiologically, at least in part, by bacterial counts and sialidase activity present in vaginal fluid at the time of infection. BV is associated with a decreased count of Lactobacillus spp. and an increased count of Gardneralla vaginalis, Bacteriodes spp., Prevotella spp., Mobiluncus spp., Peptostreptococcus species, and Mycoplasma hominus. Vaginal fluid of women infected with BV can also present elevated levels of sialidase activity. Sialidases, or neuraminidases, are enzymes that cleave alpha-ketosidic linkages between the glycosyl residues of glycoproteins, glycolipids, or colominic acids and sialic acids. Sialidases are thought to be virulence factors in several pathogenic organisms, including Corynebacterium diphtheriae, Vibrio cholerae, Streptococcus pneumoniae, group B streptococci, and many of the organisms associated with BV including Bacteriodes and Prevotella.

Increased sialidase activity among women infected with BV can create a synergistic effect during a BV infection, thereby leading to further infection and complications. Elevated sialidase activity is believed to result in the reduction or loss of sialic acid residues and subterminal sugars from cervical mucins, thereby lowering the viscosity of cervical mucus. As a result, mucus organization can be diminished, and its effectiveness as a mechanical and bacteriostatic barrier can be reduced. Removal of sialic acid residues and subterminal sugars can also leave structures in the oligosaccharide layer of vaginal epithelial cells exposed and thereby promote bacterial adhesion to the secreted mucus and the underlying epithelial glycocalyx. Such a synergy can create favorable conditions that enable certain bacteria to adhere, invade, and destroy mucosal tissue of the upper reproductive tract. In pregnant women, such an invasion can result in the release of inflammatory mediators that initiate labor. In a recent study, for example, vaginal colonization with bacteria linked to BV at concentrations greater than 10⁴ CFU/mL of vaginal fluid was associated with a twofold increased risk of preterm delivery among women in preterm labor (Krohn, et al. “Vaginal Bacteriodes species are associated with an increased rate of preterm delivery among women in preterm labor.” J. Infect. Dis. 1991 (164):88-93).

Despite the prevalence and potential dire consequences of BV, early identification and diagnosis of BV can be difficult. Several diagnoses exist for bacterial vaginosis, many of which are laborious and require extensive laboratory testing. Predominantly, clinical diagnosis of bacterial vaginosis requires the presence of three out of four criteria elements as described by Amsel: (1) thin, homogeneous, milky vaginal discharge; (2) vaginal-fluid pH greater than 4.5; (3) a positive whiff test (i.e., production of a fishy odor when 10% KOH is added to a slide containing vaginal fluid); and (4) clue cells (greater than 20% of epithelial cells with adherent bacteria) (Amsel et al. “Nonspecific vaginitis: diagnostic criteria and microbial and epidemiologic associations.” Am. J. Med. 1983 (74):14-22). An alternative diagnostic approach utilizes Gram's staining of vaginal fluid to distinguish normal vaginal flora (i.e. gram-positive rods and lactobacilli) from bacterial vaginosis flora (gram-negative morphotypes). Another alternative approach is based on the detection and measurement of sialidase activity. A commercially available test, BVBlue™, utilizes this approach, wherein a colorimetric substrate changes from yellow to blue upon enzymatic cleavage of ketosidic linkages by sialidases. The BVBlue™ test enables rapid detection of BV in a physician's office, and can thereby provide advantages over the Amsel methods and other methods requiring extensive laboratory testing.

Despite the existence of current diagnostic methods for BV, there exists a need for alternative diagnostic methods, particularly methods that do not require a physician's visit. Self-diagnostic approaches are particularly desirable given that many women infected with BV do not consult a physician. One reason for this is that many women infected with BV only present cryptic symptoms, such as an ambiguous, malodorous vaginal discharge not unlike a normal vaginal discharge. Many other women infected with BV are asymptomatic.

Thus, there is a need to address the aforementioned problems and other shortcomings associated with bacterial vaginosis. These needs and other needs are satisfied by the devices and methods of the present invention.

### SUMMARY

Disclosed herein are fibers comprising synthetic, naturally occurring polymers, or mixtures thereof, wherein the polymers are modified to electrostatically attach thereto one or more substrates. The disclosed substrates are substrates for one or more biologically active substances and upon contacting the substrates with a body fluid or a sample comprising body fluid having one or more biologically active substances contained therein, the substrate release a visual or odorous signal that the biologically active substance is present.

Further disclosed are composites that comprise at least a portion of one or more of the disclosed fibers. The composites can be used to detect the presence of one or more biologically active substances that contact the substrate. Also disclosed are articles of manufacture that either comprise, as a portion, one or more of the disclosed fibers, or a disclosed composite.

Yet further disclosed are methods for detecting the presence of a biologically active substance comprising contacting a fiber, composite, or article of manufacture as disclosed herein, with body fluid or a sample that comprises body fluid.

### BRIEF DESCRIPTION OF THE FIGURES

FIG. 1 depicts an example of a hollow fiber that can absorb and retain fluid.

FIG. 2 depicts the cross section of a coiled or spiral shaped polymer.

FIG. 3 depicts an example of a disclosed polymer yarn comprising and admixture of poly(vinylidene fluoride) and polyethylene oxide.

FIG. 4 depicts a disclosed yarn comprising polyethylene oxide.

FIG. 5 depicts a combination yarn wherein the yarn from depicted in FIG. 4 is place into the hole of the spiral fiber depicted in FIG. 2.

FIG. 6 depicts a surface modified fiber core having a plurality of anionic groups hat can electrostatically immobilize a signaling substrate Z.

FIG. 7 depicts a surface modified fiber core having a plurality of cationic groups, that can electrostatically immobilize a signaling substrate Z.

FIG. 8 depicts a surface modified fiber core comprising hydroxyl groups that has been treated with poly(glycidyl methacrylate).

FIG. 9 depicts an example of a surface modified fiber core that has a covalently attached substrate anchor wherein the substrate anchor comprises a terminal hydroxyl group.

FIG. 10 depicts an example wherein the fiber depicted in FIG. 9 is reacted with a second substrate anchor precursor to form a substrate anchor that has a higher density of anionic groups (carboxylate) capable of electrostatically immobilizing a signaling substrate.

FIG. 11 depicts an example wherein the immobilizing units are cationic units that can immobilize a signaling substrate having an anionic unit.

FIG. 12 depicts a covalently attached substrate anchor having a carboxylate

(anionic) end group that can electrostatically immobilize a signaling substrate Z having an amino unit.

FIG. 13 depicts a polysaccharide that has a first substrate anchor segment formed by reacting the polysaccharide hydroxyl groups with two moles of ethylene oxide.

FIG. 14 depicts a product wherein the polysaccharide is reacted with an omega-halo alcohol, for example, 5-bromopentanol.

FIG. 15 depicts a modified fiber core, for example, nylon 66 that has a substrate anchor electrostatically attached thereto and showing further electrostatic immobilization of a signaling substrate Z.

FIG. 16 depicts a substrate anchor electrostatically attached to a fiber core wherein the substrate anchor has further units to electrostatically immobilize a signaling substrate.

FIG. 17 depicts an example of the random spatial arrangement of substrate anchors 114 having electrostatic end groups (amino) along the surface 112 of a fiber forming a permeable skin over the core 110.

FIG. 18 depicts an example wherein N-acetyl neuraminic acid serves as a biologically active substrate that is electrostatically immbilized along the surface 112 of a fiber by the positively charged amino groups of a substrate anchor.

FIG. 19 depicts the absorbance of a standardized solution of a substrate (A) and the change in absorbance of the standardized solution of the substrate (B) after contacting the solution with a polymer having oppositely charge groups. The change in absorbance corresponds to approximately 30% of the substrate originally in solution now being electrostatically immobilized on the fiber as described in Example 1.

FIG. 20 is a photomicrograph showing the positive color change observed for the positive reaction described in Example 1.

FIG. 21 depicts the UV spectra of 0.58 mg/mL BCIN solution before (▴) and after () its incubation with a positively charged nylon fiber as described in Example 2. FIG. 22A depicts the fiber depicted in Example 2 prior to exposure to 0.06 U of sialidase from Arthrobacter ureafaciens in phosphate buffer saline (pH 5.5).

FIG. 22B depicts the fiber depicted in Example 2 turns blue after exposure to 0.06 U of sialidase from Arthrobacter ureafaciens in phosphate buffer saline (pH 5.5).

### DETAILED DESCRIPTION

Before the present polymers, fibers, compositions, substrates, composites, articles of manufacture and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting. All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which need to be independently confirmed.

Throughout the description and claims of this specification the term “comprise” and other forms of the term, such as “comprising” and “comprises,” means including but not limited to, and is not intended to exclude, for example, other elements, additives, components, integers, or steps. Thus, such terms are inclusive or open-ended transitional terms and do not exclude additional, unrecited elements, additives, components, integers, or steps. In one aspect, these terms are synonymous with “having,” “including,” “containing,” or “characterized by.”

As used herein, the terms “consisting essentially of” or “consists essentially of” are generally open-ended transitional terms, but limit the scope of a claim to the specified materials or steps and those that do not materially affect the basic and novel characteristic(s) of the claimed invention.

As used in the description and the appended claims, the singular forms “a,” “an,” and “the” include plural referents unless the context clearly dictates otherwise. Thus, for example, reference to “a nanoparticle” includes mixtures of two or more such nanoparticles. “Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed, then “less than or equal to” the value, “greater than or equal to the value,” and possible ranges between values are also disclosed, as appropriately understood by the skilled artisan. For example, if the value “10” is disclosed, then “less than or equal to 10” as well as “greater than or equal to 10” is also disclosed. It is also understood that throughout the application data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units is also disclosed. For example, if 10 and 15 are disclosed, then 11, 12, 13, and 14 are also disclosed.

“Monomer” as used herein is a molecule that can undergo polymerization thereby contributing constitutional units to the essential structure of a macromolecule, an oligomer, a block, a chain, and the like.

“Polymer” as used herein is a macromolecule comprising multiple repeating smaller units or molecules (monomers) derived, actually or conceptually, from smaller units or molecules. The polymer can be natural or synthetic.

“Copolymer” as used herein is a polymer derived from more than one species of monomer.

“Block copolymer” as used herein is a copolymer that comprises more than one species of monomer, wherein the monomers are present in blocks. Each block of the monomer comprises repeating sequences of the monomer.

“Crosslinkable polymer” as used herein is a polymer having a small region in the polymer from which at least 1-4 polymer chains may emanate, and may be formed by reactions involving sites or groups on existing polymers or may be formed by interactions between existing polymers. The small region may be an atom, a group of atoms, or a number of branch points connected by bonds, groups of atoms, or polymer chains. Typically, a crosslink is a covalent structure but the term is also used to describe sites of weaker chemical interactions, portions of crystallites, and even physical interactions such as phase separation and entanglements.

“Morphology” as used herein is to describe a form, a shape, a structure, and the like of a substance, a material, and the like as well as other physical and chemical properties (e.g. Young's Modulus, dielectric constant, etc. as described infra).

“Signaling substrate” or “recognition element” as used herein is a chemical entity that is electrostatically immobilized to the fiber and which can interact with one or more biologically active substances to indicate the presence of the one or more biologically active substances.

“Yarns” as used herein comprises a plurality of the disclosed fibers. The yarns can comprise synthetic fibers, naturally occurring fibers, or mixtures of synthetic and naturally occurring fibers.

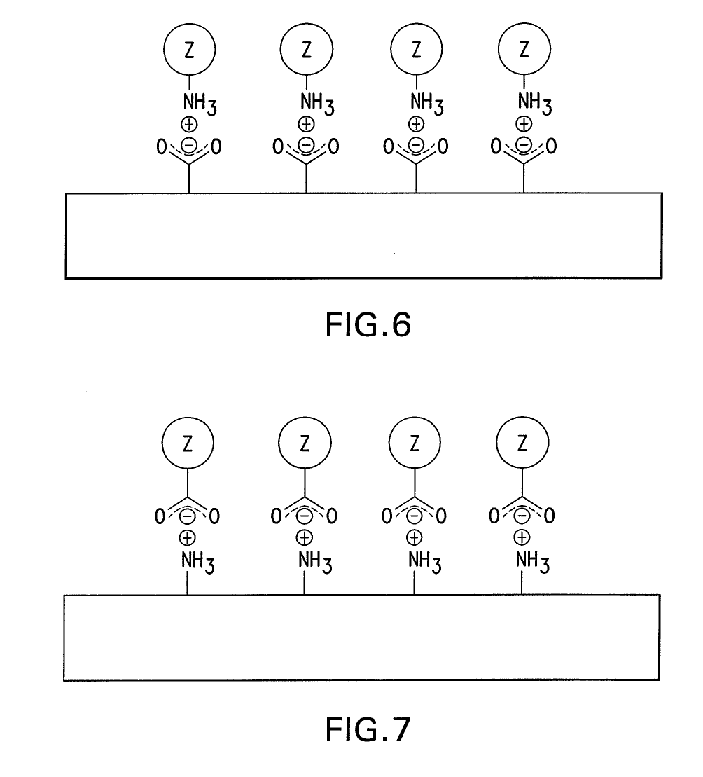

“Electrostatically attach” and “electrostatically immobilize” as used herein refers to the attachment of a signaling substrate to a fiber, yarn, spindle, filament, or bundle, or to a tether grafted to a fiber, yarn, spindle, filament, or bundle wherein the signaling substrate comprises an opposite charge from the fiber, yarn, spindle, filament, or bundle or graft thereto. FIGS. 6-18 depict non-limiting examples of fibers, fiber cores, fiber cores having attached thereto a substrate anchor, and signaling substrates electrostatically immobilized thereon.

“Composite” as used herein is a physical body that is comprised of one or more of the disclosed fibers. Non-limiting examples of composites include bandages, dressings, catamenial pads, minipads, topsheets for bandages, dressings, catamenial pads or minipads, woven or non-woven absorbent materials, and the like. (e.g. a layer or a laminate, a material, and the like) onto which a polymer or polymeric material may be deposited on or adhered to.

Some of the microorganisms responsible for Bacterial Vaginosis include Lactobacillus crispatus, Lactobacillus jensenii, Gardnerella vaginalis, Mobiluncus, Bacterocides, and Mycoplasma. The presence of these microorganisms is typically diagnosed using one of more of the following tests.

Amsel criteria (Amsel, R et al., “Nonspecific vaginitis. Diagnostic criteria and microbial and epidemiologic associations”, Am J Med (1983)74:14-22) wherein at least three of the four criteria are present.

1) Thin, white, yellow, homogeneous discharge;

2) Clue cell on microscopy;

3) pH of vaginal fluid greater than about 4.5; and

4) Release of fishy odor upon the addition of 10% potassium hydroxide.

Alternatively, a Gram stained vaginal smear with the Hay/Ison criteria (Ison, C A et al., “Validation of a simplified grading of Gram stained vaginal smears for use in genitourinary medicine clinics”, Sex Transm Infect 78:413-415 (2002)) or the Nugent criteria (Nugent, R. P. et al. “Reliability of diagnosing bacterial vaginosis is improved by a standardized method of Gram stain interpretation”. J. Clin. Microbiol. 29:297-301 (1991)) are used.

The aforementioned tests require that a sample of the vaginal fluid be taken, typically with a swab, which requires a clinical setting. Thus the above tests cannot be done at home and a rapid evaluation of symptoms provided to the patient. As such the present disclosure provides a quick, inexpensive, and consumer compatible method for determining the presence of a disease, illness, syndrome, or exposure to a microorganism.

Disclosed herein are modified fibers, composites, comprising modified fibers, articles of manufacture comprising one or more substrates, and methods for detecting the presence of a biologically active substance. The term “biologically active substance” means any molecule produced by a cell that has biological activity, for example, an enzyme, an antigen, a hormone, DNA, RNA, or fragments thereof. The biologically active substance can be present due to any circumstance, for example, the substance can be produced by a microorganism or the substance can be present due to the presence of a trauma, wound, illness, microorganism and the like. The substance can be present during normal health, but elevated due to illness and the like. The substrates disclosed herein can be modified to detect the presence of a plurality of biologically active substances, for example, in the instance wherein determining the presence of more than one biologically active substances is necessary to confirm or to establish the presence of a microorganism, illness, or other condition related to the presence of the biologically active substance.

### Fibers

The disclosed fibers provide a number of unmet needs for providing a method of signaling the presence of one or more biologically active substances while simultaneously retaining the medium (i.e., body fluid) that contains the biologically active substance, including:
a) the disclosed fibers, or the yarns formed from the disclosed fibers, provide a highly porous matrix that can hold and retain a fluid which contains one or more biologically active substances while allowing for contact between a signaling substrate and the targeted biologically active substance or substances;
b) the disclosed fibers, or the yarns formed from the disclosed fibers, can be modified to comprise various nanometer pores thereby allowing the formulator to increase or decrease the rate of liquid transport and hence, the ability of the fibers or yarns to retain fluid;
c) the disclosed fibers, or the yarns formed from the disclosed fibers, can have their surfaces modified to comprise various reactive groups that can be used to electrostatically attach one or more signaling substrates; and
d) the disclosed fibers, or the yarns formed from the disclosed fibers, can have their surfaces modified to comprise various reactive groups that can be used to chemically attach the proximal end of a tether to the fiber wherein the distal end of the tether is capable of electrostatically attaching one or more signaling substrates.

The disclosed composites provide a number of unmet needs, including:
a) providing a method for consumers to detect and/or determine the presence of a biologically active substance in human or animal body fluid;
b) providing an article of manufacture that can assist medical personnel in diagnosing the presence of a disease, syndrome, or infection, or to monitor the progress of a therapy used to cure a disease, syndrome, infection, or the like; and
c) providing a composite that can be modified by the formulator and thereby adapted for use in detecting one or more biologically active substances in need of detection.

The disclosed composites comprise a plurality of fibers or yarns comprising a plurality of fibers. The disclosed composites can have a single layer or multiple layers. The layers can comprise the same or different fibers or yarns. Each layer can be formed in a different manner, for example, one layer can be woven while other layers can comprise a woven polymer. The disclosed composites include woven or unwoven sheets or layers that can be used to amend the surface of an existing composite, for example, a disclosed composite can have a first side having an adhesive that is placed on the surface of a catamenial, inter alia, a panty liner. In this embodiment, the composite can comprise a biologically active substance signaling substrate or recognition element and utilize the absorptive properties of the catamenial to direct body fluid away from the user's skin surface.

The fibers that comprise the composites can have a diameter of from about 10 nanometers (nm) to about 1 mm (1,000 μm). As such, the disclosed fibers that comprise the disclosed composites and articles of manufacture are referred to herein as “nanofibers.” The terms “fiber” and “nanofiber” are used interchangeably and refer to the fibers that comprise the disclosed substrates. Because the body fluids that comprise the biologically active substrates can have microorganisms, toxins, and the like within the fluid, the disclosed fibers are modified to provide a high uptake rate and a high retention ability.

The disclosed fibers comprise:
a) a core having a plurality of pores for accepting and retaining a fluid;
b) an substrate anchor wherein the substrate anchor provides one or more sites for electrostatic immobilization of a signaling substrate of an active substance; and
c) a signaling substrate.

In one embodiment, the disclosed fibers comprise:
a) a core having a plurality of pores for accepting and retaining a fluid;
b) a grafted substrate anchor covalently attached to the fiber core wherein the substrate anchor provides one or more sites for electrostatic immobilization of a signaling substrate; and
c) a signaling substrate.

In another embodiment, the disclosed fibers comprise:
a) a core having a plurality of pores for accepting and retaining a fluid;
b) an substrate anchor wherein the substrate anchor provides one or more sites for electrostatic immobilization of a signaling substrate of an active substance; and
c) an enzyme substrate.

In a further embodiment, the disclosed fibers comprise:
a) a core having a plurality of pores for accepting and retaining a fluid;
b) a grafted substrate anchor covalently attached to the fiber core wherein the substrate anchor provides one or more sites for electrostatic immobilization of a signaling substrate; and
c) an enzyme substrate.

The disclosed yarns comprise:
a) a plurality of fibers, wherein a portion of the fibers are fibers having a plurality of pores for accepting and retaining a fluid;
b) a substrate anchor on at least about 5% by weight of the fibers wherein the substrate anchor provides one or more sites for electrostatic immobilization of a signaling substrate of an active substance; and
c) one or more signaling substrates electrostatically immobilized thereto.

In another embodiment, the disclosed yarns comprise:
a) a plurality of fibers, wherein the fibers comprise synthetic polymers, a portion of which fibers are fibers having a plurality of pores for accepting and retaining a fluid;
b) a substrate anchor on at least about 5% by weight of the fibers wherein the substrate anchor provides one or more sites for electrostatic immobilization of a signaling substrate of an active substance; and
c) one or more signaling substrates electrostatically immobilized thereto.

In a further embodiment, the disclosed yarns comprise:
a) a plurality of fibers, wherein the fibers comprise naturally occurring polymers or modified naturally occurring polymers, a portion of which fibers are fibers having a plurality of pores for accepting and retaining a fluid;
b) a substrate anchor on at least about 5% by weight of the fibers wherein the substrate anchor provides one or more sites for electrostatic immobilization of a signaling substrate of an active substance; and
c) one or more signaling substrates electrostatically immobilized thereto.

In a still further embodiment, the disclosed yarns comprise:
a) a plurality of fibers, wherein the fibers comprise an admixture of synthetic and naturally occurring polymers or modified naturally occurring polymers, a portion of which fibers are fibers having a plurality of pores for accepting and retaining a fluid;
b) a substrate anchor on at least about 5% by weight of the fibers wherein the substrate anchor provides one or more sites for electrostatic immobilization of a signaling substrate of an active substance; and
c) one or more signaling substrates electrostatically immobilized thereto.

The disclosed composites comprise the herein described fibers, yarns, filaments, and/or bundles that comprise one or more naturally occurring or synthetic polymers.

### Polymeric Fiber Cores

In one aspect, the disclosed fibers, yarns, spindles, and the like, comprise:
a) a core having a plurality of pores for accepting and retaining a fluid;
b) a substrate anchor wherein the substrate anchor provides one or more sites for electrostatic immobilization of a signaling substrate of an active substance; and
c) a signaling substrate.

In a further aspect, the disclosed fibers, yarns, spindles, and the like, comprise:
a) a core having a plurality of pores for accepting and retaining a fluid and one or more moieties formed by surface modification of the core that can serve as a substrate anchor for electrostatic immobilization of a signaling substrate; and
b) a signaling substrate.

As described herein, the fiber core can comprise any naturally occurring or synthetic polymer, i.e., homopolymer, copolymer, block copolymer, and the like, or mixtures of homopolymers, copolymers, block copolymers, etc. The surface of the core comprises a plurality of pores that can accept and retain a fluid, for example, a body fluid. As such, the core wicks away excess fluid from the substrate anchor which is deposited upon the surface of the core.

The fibers further comprise one or more substrate anchors. The substrate anchors can comprise a plurality of components that are serially deposited or reacted serially to form the substrate anchors. For example, as described herein the fiber cores can be electrostatically modified to provide reactive moieties, i.e., hydroxyl, carboxyl, amino moieties wherein the substrate anchors can be attached. The substrate anchors can comprise a single chemical entity which attaches to the modified core surface and which attaches at the other end to a signaling substrate.

The signaling substrate is any substrate that can react with an active substrate that is found in body fluid or which is at a higher concentration in body fluid associated with one or more diseases. The signaling substrate can react with a single active substrate, can react with a particular class of substrates, or can react with an active substrate that is formed through a biological or chemical process from a species present in body fluid when one or more microorganisms are present. The latter is the indirect method of signaling.

By this method, a substance, for example a toxin, produced by a microorganism can react with a reagent that is attached to the substrate anchor. After reacting with the reagent, another species formed from this reaction can then react with the signaling substrate.

The disclosed fiber cores can be linear solid fibers that are porous and as such can absorb and retain fluid. The disclosed fibers have pores with an average diameter of about 100,000 nm or less. The disclosed pores are alternatively referred to herein as “nanopores.” As such, the fibers are alternatively referred to herein as “nanoporous fibers.” The disclosed fibers or nanoporous fibers have one or more pores per square micrometer (μm²) of fiber surface. In one embodiment, the pores have an average diameter of from about 10 nm to about 100 mm. In another embodiment, the pores have an average diameter of from about 20 nm to about 90 nm. In a further embodiment, the pores have an average diameter of from about 30 nm to about 80 nm. In a yet further embodiment, the pores have an average diameter of from about 20 nm to about 50 nm. In a still further embodiment, the pores have an average diameter of from about 40 nm to about 90 nm. In a yet still further embodiment, the pores have an average diameter of from about 30 nm to about 70 nm. In a yet still further embodiment, the pores have an average diameter of from about 0.1 nm to about 50 nm. In another yet still further embodiment, the pores have an average diameter of from about 1 nm to about 10 nm. In a still another yet further embodiment, the pores have an average diameter of from about 5 nm to about 30 nm. As such, the pores can have any average size, for example, 1 nm, 2 nm, 3 nm, 4, nm, 5 nm, 6 nm, 7 nm, 8 nm, 9 nm, 10 nm, and the like. The average size can also be a fraction of a whole number, for example, 3.6 nm, 4.1 nm, and 5.9 nm. The average size can be reported as a whole number, for example, 3.6 nm and 4.1 nm can both be measured as 4 nm. The actual size of the pores can be any size within the range of less than or equal to about 100,000 nm.

The disclosed pores can have irregular profiles, for example, they can be oval instead of circular. The average diameter of the pores is therefore determined from the areas of the pores assuming the pores have perfect circular profiles. The pores can be formed during the manufacture and processing of the fibers, for example, linear solid fibers produced by a process such as that disclosed in U.S. Pat. No. 7,097,904 which is included herein by reference in its entirety.

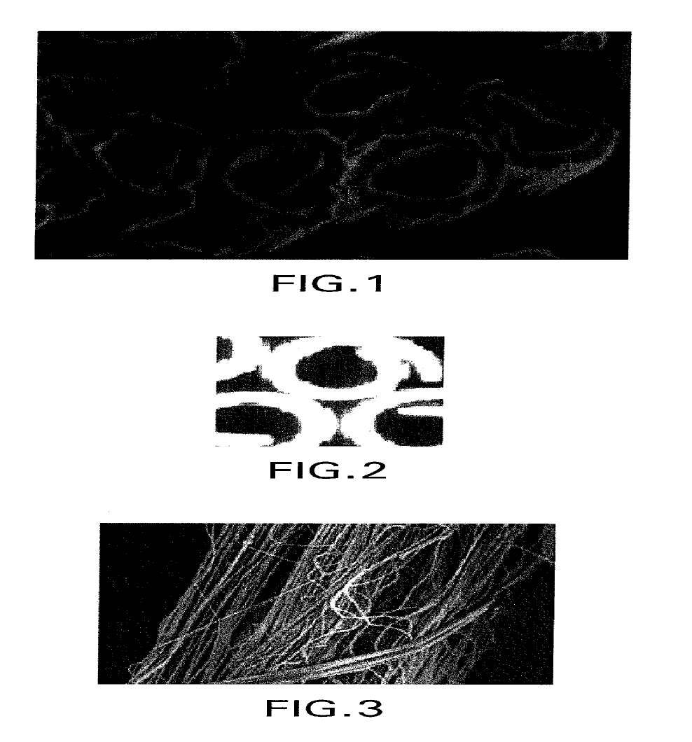

The fibers can also be shaped polymer fibers, for example, star shaped or spiral along the long axis of the fiber. FIG. 1 provides an example of a hollow fiber that can absorb and retain fluid. The composite can be formed entirely of shaped polymer fibers or the composite can comprise an admixture of shaped and unshaped, shaped and porous, or fibers having a plurality of different shapes including lengths.

The disclosed fibers have a surface area of from about 0.01 m²/g to about 5 m²/g. In one embodiment, the disclosed fibers have a surface area of from about 0.1 m²/g to about 4.2 m²/g. In another embodiment, the disclosed fibers have a surface area of from about 1 m²/g to about 4.2 m²/g. In a further embodiment, the disclosed fibers have a surface area of from about 1.2 m²/g to about 4.2 m²/g. In a still further embodiment, the disclosed fibers have a surface area of from about 1.5 m²/g to about 4 m²/g. In a yet further embodiment, the disclosed fibers have a surface area of from about 1.7 m²/g to about 3.8 m²/g. In a still yet further embodiment, the disclosed fibers have a surface area of from about 1 m²/g to about 3 m²/g. In a further yet another embodiment, the disclosed fibers have a surface area of from about 1.5 m²/g to about 3.5 m²/g.

The disclosed fiber can have a strength of from about 0.01 centinewton/decitex (cN/dtex) to about 10 cN/dtex. In one embodiment, the disclosed fibers can have a strength of from about 0.1 cN/dtex to about 8 cN/dtex. In another embodiment, the disclosed fibers can have a strength of from about 0.1 cN/dtex to about 5 cN/dtex. In a further embodiment, the disclosed fibers can have a strength of from about 1 cN/dtex to about 5 cN/dtex. In a yet further embodiment, the disclosed fibers can have a strength of from about 0.1 cN/dtex to about 3 cN/dtex. In a still further embodiment, the disclosed fibers can have a strength of from about 0.1 cN/dtex to about 2 cN/dtex. In a still yet further embodiment, the disclosed fibers can have a strength of from about 0.5 cN/dtex to about 2 cN/dtex. As such, the fibers can have any strength, for example, 1 cN/dtex, 2 cN/dtex, 3 cN/dtex, 4, cN/dtex, 5 cN/dtex, 6 cN/dtex, 7 cN/dtex, 8 cN/dtex, 9 cN/dtex, 10 cN/dtex, and the like. The average size can also be a fraction of a whole number, for example, 3.6 cN/dtex, 4.1 cN/dtex, and 5.9 cN/dtex. The average size can be reported as a whole number, for example, 3.6 cN/dtex and 4.1 cN/dtex can both be measured as 4 cN/dtex. The actual size of the pores can be any size within the range of form about 0.01 cN/dtex to about 10 cN/dtex.

Shaped fibers can be hollow, star-shaped, spiral wherein the spiral winds along the long axis of the fiber, or any other shape. The shaped fibers can have differential surface properties, for example, the outer surface of a shaped polymer can have a plurality of nanopores while the inner surface is essentially smooth. Likewise, both the inner and outer surfaces of a shaped fiber can have a plurality of nanopores. In addition, the average size of the nanopores that comprise a shaped polymer can be the same of different on the inner and outer surfaces. FIG. 2 depicts the cross section of a coiled or spiral shaped polymer.

### Yarns

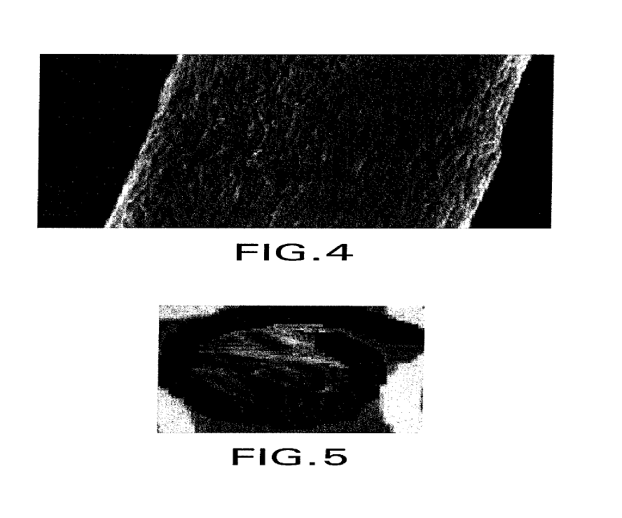

The disclosed yarns are highly absorbent. The yarns can be absorbent because they comprise the disclosed porous fibers, or alternatively, the yarns can be fabricated from fibers that do not comprises pores, but instead are woven together in a manner such that the fiber entanglement produces a network of interstices or void spaces (reticules) that can absorb and retain fluid. Alternatively the yarns can comprise both fibers that comprise pores and fiber that do not comprise pores. In another embodiment, the yarns comprise an admixture of shaped or unshaped fibers. FIG. 3 depicts an example of a polymer yarn comprising and admixture of poly(vinylidene fluoride) and polyethylene oxide. FIG. 4 depicts a yarn comprising polyethylene oxide. FIG. 5 depicts a combination yarn wherein the yarn from depicted in FIG. 4 is place into the hole of the spiral fiber depicted in FIG. 2. This arrangement provides a yarn wherein liquid can pass through the gaps in the spiral fiber wall into the highly absorbent yarn.

### Polymers

The disclosed fibers can comprise any polymeric material, for example, the fibers can be entirely synthetic or entirely naturally occurring. In addition, the fibers can be a blend of both synthetic and naturally occurring fibers. By the term “naturally occurring fiber” is meant the source of the fiber or a component of the fiber is derived from a plant or an animal, inter alia, cotton, wool, and the like. The natural fibers can be processed in a manner that only a component of the fiber is used or the naturally occurring fiber can be derivatized by any process known to the artisan, for example, cellulosic material can be chemically modified to have desired properties, inter alia, increase or decreased hydrophobicity or increased or decreased hydrophilicity.

### 1. Synthetic Polymers

The following are non-limiting examples of synthetic polymers that can comprise the disclosed fibers. Block co-polymers comprising a hydrophilic block and a hydrophobic block, for example, polymers wherein:

a) the hydrophilic block comprises one or more of the following:
i) polyalkylene glycols, for example, polyethylene glycol, polypropylene glycol, and the like;
ii) polyvinyl pyrrolidone and derivatives thereof;
iii) naturally occurring, synthetic, or modified polysacharrides;
iv) peptides and/or proteins; and
v) other hydrophilic units, oligomers, homopolymers, or copolymers; and

b) the hydrophobic block comprises one or more of the following:
i) lactide, glycolide, caprolactone, and mixtures thereof,
ii) polyester, polyhydroxy acids, polyanhydrides, polyorthoesters, polyetheresters, polyesteramides, polyphosphazines, polyphosphoesters, polyphosphates, polyphosphonates, polycarbonates, polyorthocarbonates, polyamides, or copolymers thereof.

The fibers can comprise one or more homopolymers or copolymers chosen from:
i) poly(lactide)-co-(polyalkylene oxide);
ii) poly(lactide-co-glycolide)-co-(polyalkylene oxide);
iii) poly(lactide-co-caprolactone)-b-(polyalkylene oxide);
iv) poly(lactide-co-glycolide-co-caprolactone)-b-(polyalkylene oxide);
v) poly(lactide)-co-(polyvinyl pyrrolidone);
vi) poly(lactide-co-glycolide)-co-(polyvinyl pyrrolidone);
vii) poly(lactide-co-caprolactone)-b-(polyvinyl pyrrolidone);
viii) poly(lactide-co-glycolide-co-caprolactone)-b-(polyvinyl pyrrolidone);
ix) poly(lactide);
x) poly(lactide-co-glycolide);
xi) poly(lactide-co-caprolactone);
xii) poly(lactide-co-glycolide-co-caprolactone);
xiii) poly(glycolide-co-caprolactone);
xiv) poly(caprolactone);
xv) polyesters; for example, polyethylene, polypropylene;
xvi) polyurethanes;
xvii) polyhydroxy acids;
xviii) polyanhydrides;
xix) polyorthoesters,
xx) polyetheresters,
xxi) polyesteramides,
xxii) polyphosphazines,
xxiii) polyphosphoesters,
xxiv) polyphosphates,
xxv) polyphosphonates,
xxvi) polycarbonates,
xxvii) polyorthocarbonates,
xxviii) polyamides.

In one embodiment, the fibers comprise a polymer chosen from poly(ethylene terephthalate), nylon, polyethyleneimine, poly(vinylidene fluoride), polyethylene, polysiloxane, polystyrene, polyethylene glycol, or a mixture thereof.

In another embodiment, the fibers comprise a polymer chosen from polystyrene-polyvinyl pyridine, polystyrene-polybutadiene, polystyrene-hydrogenated polybutadiene, polystyrene-polyisoprene, polystyrene-hydrogenated polyisoprene, polystyrene-poly(methyl methacrylate), polystyrene-polyalkenyl aromatics, polyisoprene-poly(ethylene oxide), polystyrene-poly(ethylene propylene), poly(ethylene oxide)-polycaprolactones, polybutadiene-poly(ethylene oxide), polyisoprene-poly(ethylene oxide), polystyrene-poly(t-butyl methacrylate), poly(methyl methacrylate)-poly(t-butyl methacrylate), poly(ethylene oxide)-poly(propylene oxide), polystyrene-poly(t-butylacrylate), and polystyrene-poly(tetrahydrofuran).

### 2. Natural Polymers

The following are non-limiting examples of naturally occurring polymers that can comprise the disclosed fibers and yarns.

The disclosed fibers and yarns can comprise polysaccharides, for example, cellulosic polymers. Non-limiting examples include cellulose and variations thereof or chemically modified cellulose, for example, cellulose esters, such as cellulose mono-acetate, cellulose di-acetate, cellulose tri-acetate, cellulose propionate, cellulose butyrate, cellulose acetobutyrate, cellulose acetopropionate, cellulose nitrate, and the like, and mixtures thereof.

Naturally occurring fibers obtained from plants include cotton, kapok, jute, flax, ramie, sisal, agave, kenaf, rattan, soybean fiber, and hemp. Naturally occurring fibers obtained from animals or insects include silk, wool, angora, mohair and alpaca.

The molecular weight of the polymers that comprise the disclosed fibers, yarns, and bundles can be from about 500 daltons to about 2,000,000 daltons. In one embodiment, the average molecular weight of the polymer can be from about 2,000 daltons to about 200,000 daltons. In another embodiment, the average molecular weight of the polymer can be from about 500 daltons to about 5,000 daltons. Wherein a further aspect of this embodiment comprises copolymers wherein the polymer has an average molecular weight of from 500 daltons to 1,500 daltons. In a yet further embodiment, the molecular weight of the polymer can be from about 1,000 daltons to about 200,000 daltons. In another further embodiment, the molecular weight of the polymer can be from about 4,000 daltons to about 150,000 daltons. And in a yet further embodiment, the molecular weight of the polymer can be from about 4,000 daltons to about 100,000 daltons. The molecular weight of the polymer of the copolymers of the present disclosure can be from about 100 daltons to about 100,000 daltons. In another embodiment, the molecular weight of the polymer can be from about 100 daltons to about 40,000 daltons. In yet another embodiment, the molecular weight of the polymer can be from about 100 daltons to about 8,000 daltons. A further embodiment comprises a polymer having a molecular weight of from about 1,000 daltons to about 8,000 daltons. A yet another further embodiment comprises a polymer having a molecular weight of from about 1,000 daltons to about 6,000 daltons. In a still yet another embodiment comprises a polymer having a molecular weight of from about 10,000 daltons to about 100,000 daltons. In a still yet further embodiment comprises a polymer having a molecular weight of from about 5,000 daltons to about 50,000 daltons. Another further embodiment comprises a polymer having a molecular weight of from about 3,000 daltons to about 12,000 daltons. A still further embodiment comprises a polymer having a molecular weight of from about 400 daltons to about 4,000 daltons.

The polymer average molecular weights can be obtained be Gel Permeation Chromatography (GPC), for example, as described by L. H. Sperling of the Center for Polymer Science and Engineering & Polymer Interfaces Center, Materials Research Center, Department of Chemical Engineering and Materials Science and Engineering Department, Lehigh University, 5 E. Packer Ave., Bethlehem, Pa. 18015-3194, as first described in: ACS Division of Polymeric Materials: Science and Engineering (PMSE), 81:569 (1999).

Alternatively the molecular weights can be described by their measured Inherent Viscosity (IV) as determined by capillary viscometry. Molecular weights of the polymers described herein can be about 0.05 dL/g to about 2.0 dL/g wherein dL is deciliter. In another embodiment the inherent viscosity can be from about 0.05 dL/g to about 1.2 dL/g. In a further embodiment the inherent viscosity can be form about 0.1 dL/g to about 1.0 dL/g. A yet further embodiment of the polymers and copolymers of the present disclosure can have an inherent viscosity of from about 0.1 dL/g to about 0.8 dL/g. And yet another embodiment of the polymers of the present disclosure can have an inherent viscosity of from about 0.05 dL/g to about 0.5 dL/g. Alternatively, the formulator can express the inherent viscosity in cm³/g if convenient.

### Substrate Anchors

Disclosed herein are fibers having an electrostatically immobilized signaling substrate attached thereto. In a first aspect, the fiber cores can have their surfaces modified in a manner that allows for the signaling substrate to be electrostatically immobilized directly to a portion of the surface modified fiber core. As it relates to this aspect, the fragment of the fiber core that results from surface modification that serves to electrostatically immobilize the signaling substrate is defined herein as the “substrate anchor.

As such, this aspect of the disclosed fibers, comprise:
a) a core having a plurality of pores for accepting and retaining a fluid and one or more moieties formed by surface modification of the core that can serve as a substrate anchor for electrostatic immobilization of a signaling substrate; and
b) a signaling substrate.

FIG. 6 depicts a surface modified fiber core having a plurality of anionic groups, for example, carboxylate groups that can electrostatically immobilize a signaling substrate Z having a cationic group, i.e., amino groups. FIG. 7 depicts a surface modified fiber core having a plurality of cationic groups, for example, amino groups that can electrostatically immobilize a signaling substrate Z having a anionic group, i.e., carboxylate groups.

As it relates to this aspect of the disclosed fibers, the fiber core surface can be modified by any method chosen by the formulator, for example, by chemical modification or by air corona/plasma treatment.

A further aspect of the disclosed fibers relates to fibers comprising a substrate anchor that is not a part of the original fiber core but which is attached to the fiber core surface by one or more of the methods disclosed herein below. As such, this aspect of the disclosed fibers comprises:
a) a core having a plurality of pores for accepting and retaining a fluid;
b) a substrate anchor attached to the core wherein the substrate anchor provides one or more sites for electrostatic immobilization of a signaling substrate of an active substance; and
c) a signaling substrate.

### Methods of Attachment of Substrate Anchors to Fiber Cores

The disclosed fibers comprise a substrate anchor that is attached to the disclosed core by one or more methods. For example, the surfaces of the disclosed polymer fibers, yarns, or bundles can be modified to comprise one or more units, i.e., reactive moieties such as amino, hydroxyl, carboxylate, and the like, that can:
a) covalently attach one or more substrate anchor;
b) electrostatically attach one or more substrate anchors; or
c) a combination thereof.

### 1. Covalent Attachment to Surface Modified Fiber Cores

The disclosed fiber cores can be modified to comprise moieties that can covalently react with a compound that forms the substrate anchor and that is subsequently capable of electrostatically immobilizing one or more signaling substrates. The fiber cores can be modified by any method chosen by the formulator, for example, by chemical modification or by air corona/plasma treatment. The substrate anchors can be formed by reacting the fiber core with a single species, for example, a preformed polymer such as poly(glycidyl methacrylate), or the substrate anchors can be built up by reacting a first chemical species (substrate anchor precursor) with the fiber core, followed by subsequent reaction either with one or more other substrate anchor precursors or with an additional amount of the first substrate anchor precursor chemical species. As such, the substrate anchors can be formed in one step or by a series of chemical reactions.

In one embodiment, the disclosed polymeric fibers can be chemically modified to provide hydroxyl groups that can react to covalently attach the substrate anchor. For example, poly(ethylene-co-terephthalate) can be treated with strong base as shown in the scheme below:

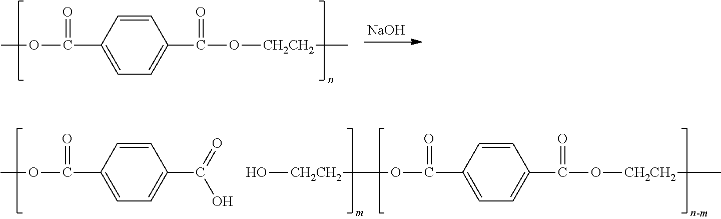

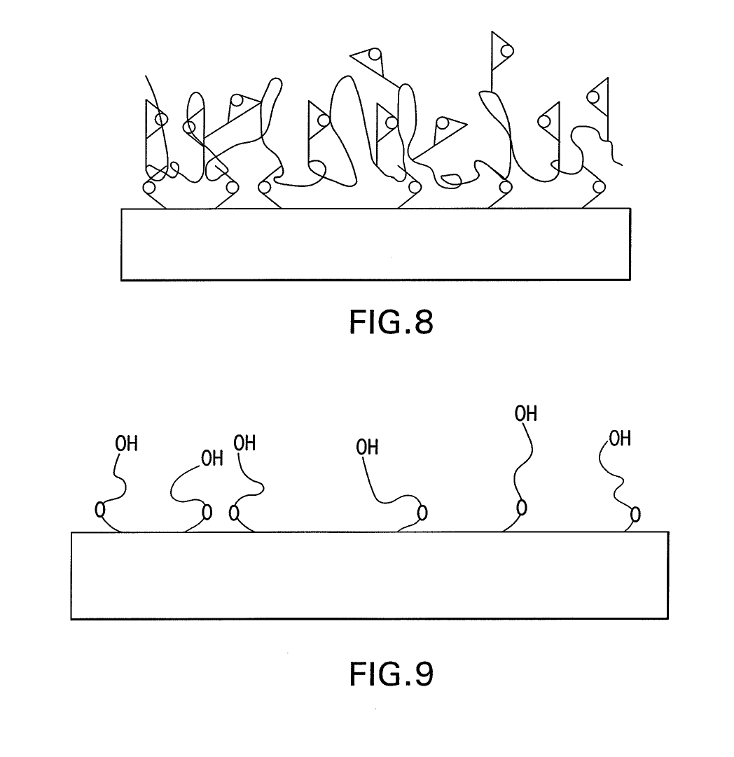

to provide hydroxyl and/or carboxylate groups that can further react with a substrate anchor precursor to form a covalently attached substrate anchor. One example of covalently attached substrate anchor is poly(glycidyl methacrylate) (PGMA) wherein the epoxy units are used to attach the substrate anchor to the polymer as described in U.S. Pat. No. 7,261,938 and U.S. Pat. No. 7,026,014 included herein by reference in their entirety. FIG. 8 depicts a surface modified fiber core comprising hydroxyl groups that has been treated with poly(glycidyl methacrylate). In this example, the surface hydroxyl groups of the modified fiber core react with epoxide units of the poly(glycidyl methacrylate) to covalently attach the polymer to the surface of the fiber core. The remaining epoxide units can then further react with either a second substrate anchoring unit capable of electrostatically immobilizing a signaling substrate or the epoxide group can be chemically modified to be a unit capable itself of electrostatically immobilizing a signaling substrate.

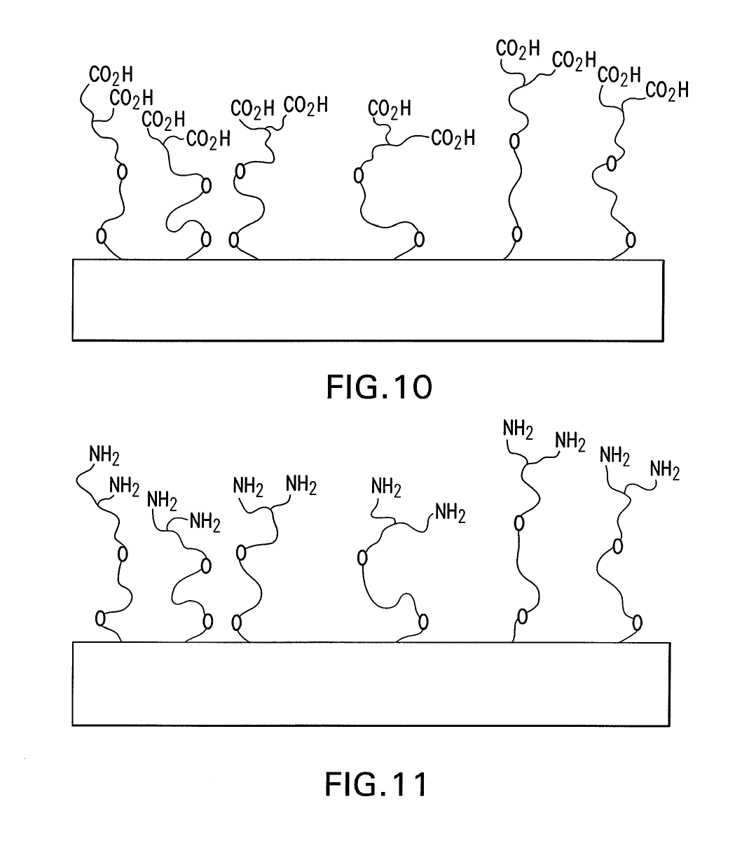

FIG. 9 depicts an example of a surface modified fiber core that has a covalently attached substrate anchor wherein the substrate anchor comprises a terminal hydroxyl group. FIG. 10 depicts an example wherein the fiber depicted in FIG. 9 is reacted with a second substrate anchor precursor to form a substrate anchor that has a higher density of anionic groups (carboxylate) capable of electrostatically immobilizing a signaling substrate having a cationic unit. FIG. 11 depicts an example wherein the immobilizing units are cationic units that can immobilize a signaling substrate having an anionic unit.

In another embodiment, the disclosed polymeric fibers can be chemically modified to provide amino groups that can react to covalently attach the substrate anchor. For example, nylon 66 can be treated with strong base as shown in the scheme below:

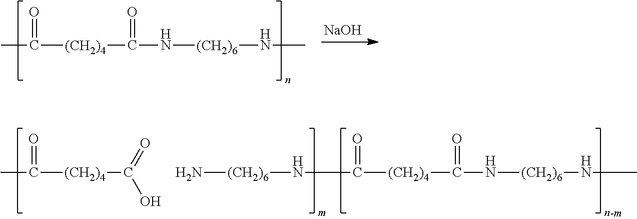

to provide amino groups that can further react with a substrate anchor precursor to form a covalently attached substrate anchor.

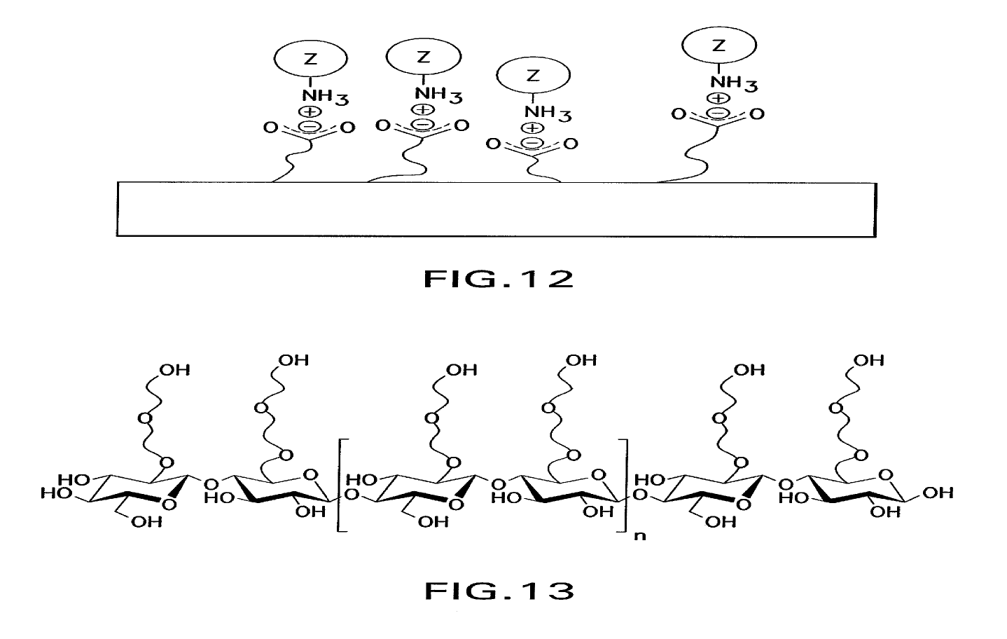

FIG. 12 depicts a covalently attached substrate anchor having a carboxylate (anionic) end group that can electrostatically immobilize a signaling substrate Z having an amino unit.

### 2. Substrate Anchor Attachment by Grafting to Fiber Cores

The disclosed fiber cores can be modified by having substrate anchors attached onto groups that are present. As such, the substrate anchor is formed by grafting onto the fiber core. The term “graft” or “grafting” refers to a process wherein one or more materials can be affixed to the fiber core by reaction of a reactive moiety present on the surface of the fiber core with another reagent that forms a chain of at least one carbon and includes at least one reactive unit for modification to a unit capable of electrostatically immobilizing a signaling substrate or, alternatively, can serve as a point for further extension of the grafted chain. The grafting can be accomplished by reacting the fiber core with a molecule that can self polymerize, for example, grafting using ethylene oxide that can form ethyleneoxy chains or with aziridine (ethyleneimine) that can form ethyleneamino chains. In addition, the grafting unit can be a monomer, for example, vinyl alcohol that can self polymerize to form polyvinyl alcohol chains covalently attached to the fiber core.

Naturally occurring polysaccharides are one example of fiber cores that comprise a plurality of units (hydroxyl groups) that can serve as a point for attachment of a substrate anchor by grafting. FIG. 13 depicts a polysaccharide that has a first substrate anchor segment formed by reacting the polysaccharide hydroxyl groups with two moles of ethylene oxide. The addition of the ethylene oxide can be done stepwise so as to react with selected hydroxyls or the reaction can be random wherein the length of the each polyoxyalkylene chain may be different. In addition, the formulator by employing techniques known to those skilled in the art can target the addition of the grafting units to any or all of the hydroxyls available for reaction.

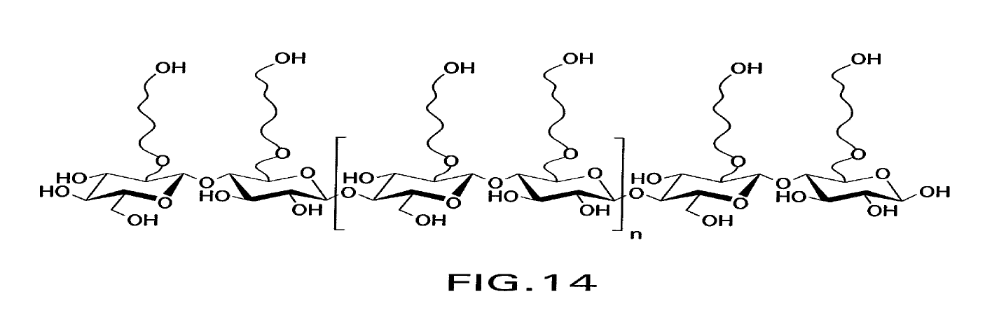

In another embodiment, the fiber cores having reactive groups can be reacted with a single chemical species to provide attachment of a first substrate anchor. For example, FIG. 14 depicts a product wherein the polysaccharide is reacted with an omega-halo alcohol, for example, 5-bromopentanol. The terminal hydroxyl group on the grafted chain can be converted to a unit that can serve as a site of electrostatic immobilization of a signaling substrate, for example, an amino group or carboxylate group. Alternatively, the terminal hydroxyl group can serve a point wherein the first substrate anchor is reacted with one or more second substrate anchors to lengthen the substrate anchor chain.

In another example, a fiber core comprising a plurality of amino groups can be reacted with a substrate anchor precursor such as an acid chloride to form an amide bond or with an alkyl halide precursor thereby forming an amino bond.

### 3. Electrostatic Attraction of Substrate Anchors to Fiber Cores

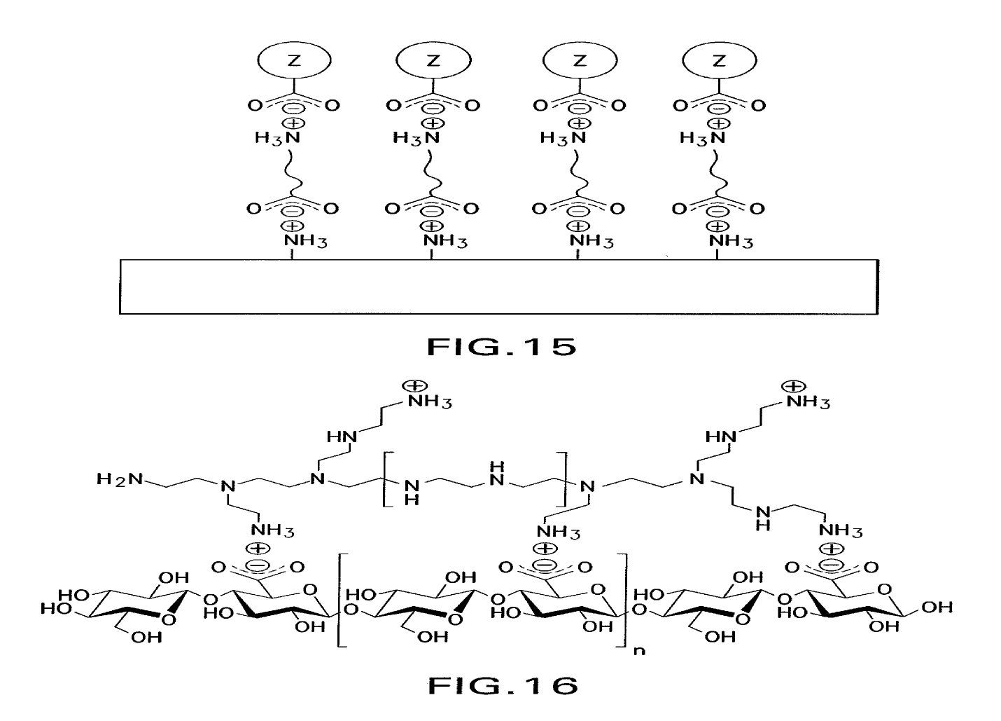

The disclosed fiber cores can have the substrate anchors attached thereto by electrostatic attraction. For example a modified fiber core or a fiber core comprising reactive units can have the substrate anchors attached electrostatic immobilization rather than being covalently attached. FIG. 15 depicts a modified fiber core, for example, nylon 66 that has a substrate anchor electrostatically attached thereto and showing further electrostatic immobilization of a signaling substrate Z.

In addition, the substrate anchors can be comprise a polymeric material that can be electrostatically attached to the fiber core using the same units that can electrostatically immobilize a signaling substrate. For example, the modified fiber core can be treated with a polyethyleneimine (PEI), a portion of which is represented by the formula:

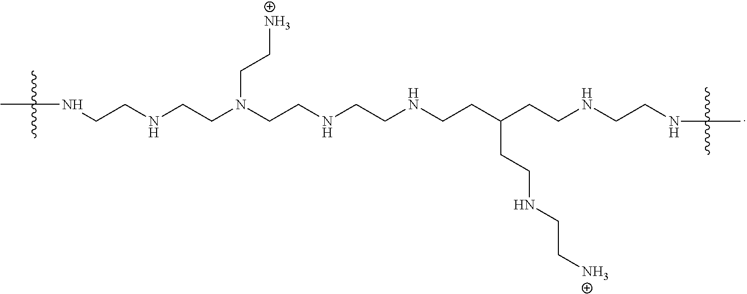

Once the PEI is electrostatically attached to the fiber core, a signaling substrate can be attached thereto. FIG. 16 depicts a substrate anchor electrostatically attached to a fiber core wherein the substrate anchor has further units to electrostatically immobilize a signaling substrate. Non-limiting examples of substrate anchors according to this aspect include polyethyleneimines available in varying molecular weights and degree of branching, for example, PEI 189, PEI 600, PEI 1200, and PEI 1800 available from BASF under the tradename LUPASOL™.

The substrate anchors provide a coating over the fiber cores which allow fluids to pass through and become collected in the pores of the fiber cores. As the fluid passes through the coating, the signaling substrate can come into contact with one or more biologically active substances that are indicative of the presence of one or more microorganisms. For example, sialidase enzyme which is present in increased amounts due to the presence of microorganisms that are related to bacterial vaginosis.

The disclosed coatings can have a thickness of from about 1 nanometers (nm) to about 25 nm. In one embodiment, the coating has a thickness of from about 5 nm to about 20 nm. In another embodiment, the coating has a thickness of from about 7 nm to about 15 nm. In a further embodiment, the coating has a thickness of from about 12 nm to about 20 nm. In a yet further embodiment, the coating has a thickness of from about 3 nm to about 10 nm. In a sill further embodiment, the coating has a thickness of from about 10 nm to about 20 nm.

### Signaling Substrates

The disclosed fibers comprise one or more signaling substrates attached by electrostatic immobilization. The signaling substrates can indicate the presence of a particular biologically active component or there can be a plurality of substrates such that more than one biologically active component can be detected. In another aspect, two different signaling substrates can be present to confirm the presence of a particular biologically active component, especially in cases wherein more than one microorganism may be present or wherein the sensitivity of the detection level must be increased.

The disclosed fibers can be used in forming substrates and articles of manufacture that provide a chemical signal in response to exposure to a biologically active compound. The chemical signal can have any form desired by the formulator, for example, a color change, an odor change, and the like. One aspect of the disclosed signaling substrates and articles of manufacture relates to the change in the substrate color in response to exposure to a particular biological substance, inter alfa, body fluid. The color change can be effected because a dye molecule is released by the presence of the biological substance, or the wavelength emitted by the chromophore may be changed because of a chemical change to the chromophore due to the biological substance, for example, the chromophore undergoes a redox reaction.

One embodiment of the color change aspect of the disclosed substrates relates to the detection of the presence of a particular enzyme. As such, the substrate can be used to detect whether one or more species of pathogen is present in a body fluid or whether the biological substance itself is harmful. For example, it is known that microorganisms contain a variety of enzymes not typically found in normal, healthy body fluids. Moreover, these enzymes catalyze reactions that are also not native to a healthy host. Therefore, advantage can be taken of this fact when utilizing the disclosed methods, processes, substrates, articles of manufacture and the polymeric materials that comprise the substrates and articles of manufacture.

For example, one enzyme that is produced by microorganisms found to cause bacterial vaginosis is sialidase. This enzyme is also known as neuramidinase. Neuraminidase cleaves terminal neuraminic acid (sialic acid) residues from carbohydrate moieties on the surfaces of infected cells. In the case of viral infections, this promotes the release of progeny viruses from infected cells. Neuraminidase is also a virulence factor for many bacteria including Bacteroides fragilis and Pseudomonis aeruginosa. Nueramidase cleaves the terminal sialic acid residue from the ganglioside-GM1 (a modulator of cell surface and receptor activity) converting it into a sialo-GM1 to which its type 4 pilli (attachment factors) bind preferentially.

N-acetylneuraminic acid can be modified to form a substrate that can be used to detect the presence of sialidase enzymes produced by microorganisms. For example, N-acteylneuraminic acid can be modified to have the general formula:

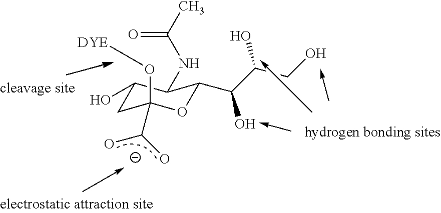

which can release a dye when acted upon by the enzyme as depicted in Scheme I.

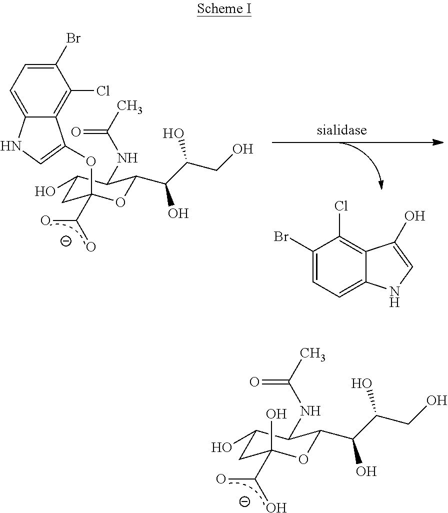

In this example, when 5-acetamido-2-(5-bromo-4-chloro-1H-indol-3-yloxy)-4-hydroxy-6-((1R,2R)-1,2,3-trihydroxypropyl)tetrahydro-2H-pyran-2-carboxylic acid is contacted with sialidase, a biologically active substance, the dye 5-bromo-4-chloro-1H-indol-3-ol is released. Typically, the substrates that can release a signal have one or more moieties that facilitate binding of the substrate to the polymer matrix. In the case of N-acetylneuraminic acid, there is an electrostatic immobilization site (i.e., carboxylate moiety) and a hydrogen bonding site.

In another embodiment, the substrate can release an odorous molecule, for example, a substrate having the general formula:

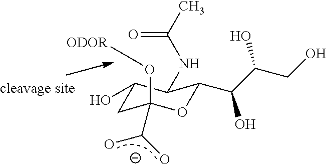

which can release an odorous molecule according to Scheme II herein below.

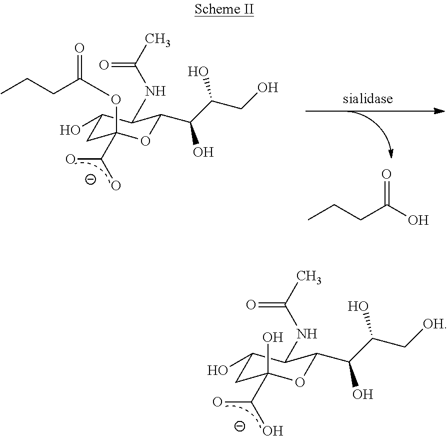

In this example, butyric acid is released; however, any fragrance raw material capable of being released by the action of sialidase can be used.

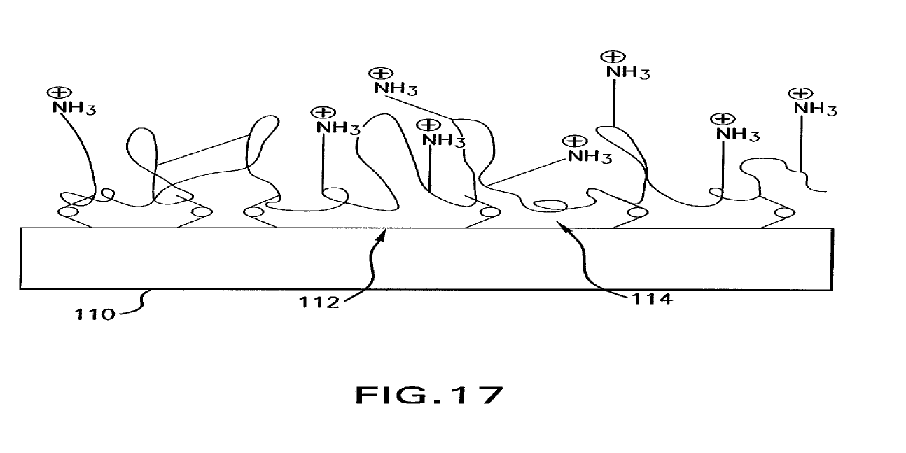

FIG. 17 depicts an example of the random spatial arrangement of substrate anchors 114 having electrostatic end groups (amino) along the surface 112 of a fiber forming a permeable skin over the core 110. This random spatial arrangement allows for more efficient capture of a biologically active component.

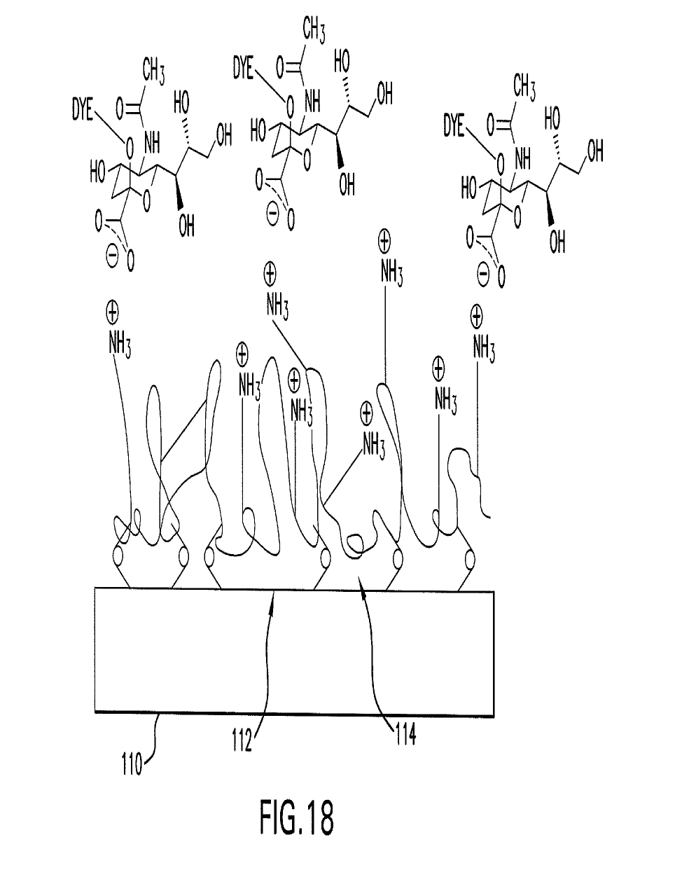

FIG. 18 depicts an example wherein N-acetyl neuraminic acid serves as a biologically active substrate that is electrostatically immbilized along the surface 112 of a fiber by the positively charged amino groups of a substrate anchor. In this embodiment, a fluid comprising a biologically active substance passes through the permeable skin 114 wherein enzymes, such as sialadase, interact with the biologically active substrate thereby releasing a color change and wherein the balance of the fluid is drawn onto the fiber core 110 where it is retained by the surface nanopores or by the interstices that exist when the core comprised a yarn or bundle.

### Composites

Disclosed herein are composites that comprise one or more of the disclosed fibers. The composite can be entirely formed from the disclosed fibers or can be formed from a mixture of the disclosed fibers and synthetic polymer fibers, naturally occurring polymer fibers, or a mixture of synthetic polymer fibers and naturally occurring fibers.

The disclosed composites can be fabricated entirely from the modified fibers disclosed herein or from a combination of modified fibers and synthetic or naturally occurring polymers. The composites can be thin topsheets that are placed on a feminine hygiene article or wound dressing that comes into contact with body fluids. The composites can be woven or nonwoven. The composites can be highly absorbent or the composites can allow for liquid transfer from the user to a second composite that absorbs and retains the fluid.

In another embodiment, the composite can be a strip comprising the disclosed fibers that is spaced at a distance on a continuous roll of material For example, a roll of material that serves as a dressing for a wound or as a swab during surgery. The fibers in this embodiment can be embedded at a distance from one another, for example, 1 to 10 inches apart. Once used, the material that came into contact with body fluid can be observed and the presence of a positive result can serve as an indication that a particular biologically active substance is present in the wound, site of trauma, or surgical incision.

The disclosed composites can be incorporated into a diaper, training pants, other child care products, infant care products, adult care products, feminine care products and the like wherein there is a need to detect the presence of a biologically active substance. Further disclosed are composites as they are applied to absorbent articles. The composites disclosed herein which comprise a mixture of fiber-types wherein only a portion of the fibers are fibers as disclosed herein that can electrostatically immobilize one or more substrates for detecting a biologically active substance, are also suitable for use as another type of absorbent article, such as a feminine care pad, an incontinence garment, training pants, pre-fastened or re-fastenable diaper pants, a wound dressing or a nursing pad. Further, the disclosed composites are not limited to application on absorbent articles. For example, the disclosed composites can be used on skin-contacting substrates such as tissues, wet (pre-moistened) wipe materials and cosmetic pads (such as for cleansing or buffing).

### Methods

Disclosed herein are methods for detecting the presence of a biologically active substance. The substance can be a chemical entity that is uniquely produced by a microorganism. The substance can be a chemical entity produced by the body in response to the presence of a microorganism. The substance can be a chemical entity produced in excess by the body in response to the presence of a microorganism. The substance can be a chemical entity produced by the body in response to trauma. The substance can be a chemical entity produced in excess by the body in response to trauma. The substance can be a chemical entity that indicates the presence of an illness, disease, or syndrome.

The disclosed methods comprise, contacting a human or a mammal with one or more of the disclosed fibers wherein the fibers comprise an electrostatically immobilized biological substrate that indicates the presence of one or more biologically active substances.

The disclosed methods further comprise, contacting a human or a mammal with a composite comprising one or more of the disclosed fibers wherein the fibers comprise an electrostatically immobilized biological substrate that indicates the presence of one or more biologically active substances.

The disclosed methods also comprise, contacting a human or a mammal with an article of manufacture comprising one or more of the disclosed fibers wherein the fibers comprise an electrostatically immobilized biological substrate that indicates the presence of one or more biologically active substances.

The disclosed methods yet further comprise, contacting a human or a mammal with an article of manufacture comprising one or more composites which comprise one or more of the disclosed fibers wherein the fibers comprise an electrostatically immobilized biological substrate that indicates the presence of one or more biologically active substances.

The disclosed methods relate to a method for determining the presence of a microorganism in vaginal fluid comprising, contacting vaginal fluid with one or more of the disclosed fibers wherein the fibers comprise an electrostatically immobilized biological substrate that indicates the presence of one or more biologically active substances produced by a microorganism associated with bacterial vaginosis.

The disclosed methods relate to a method for determining the presence of a microorganism in vaginal fluid comprising, contacting vaginal fluid with a composite that comprises one or more of the disclosed fibers wherein the fibers comprise an electrostatically immobilized biological substrate that indicates the presence of one or more biologically active substances produced by a microorganism associated with bacterial vaginosis.

The disclosed methods relate to a method for determining the presence of a microorganism in vaginal fluid comprising, contacting vaginal fluid with an article of manufacture that comprises one or more of the disclosed fibers wherein the fibers comprise an electrostatically immobilized biological substrate that indicates the presence of one or more biologically active substances produced by a microorganism associated with bacterial vaginosis.

The disclosed methods relate to a method for determining the presence of a microorganism in vaginal fluid comprising, contacting vaginal fluid with an article of manufacture that comprises one or more composites that comprise one or more of the disclosed fibers wherein the fibers comprise an electrostatically immobilized biological substrate that indicates the presence of one or more biologically active substances produced by a microorganism associated with bacterial vaginosis.

The disclosed methods relate to a method for determining the presence of a microorganism in vaginal fluid comprising, contacting vaginal fluid with an article of manufacture that comprises one or more of the disclosed fibers wherein the fibers comprise an electrostatically immobilized biological substrate that indicates the presence of one or more biologically active substances produced by a microorganism associated with bacterial vaginosis.

The disclosed methods relate to a method for determining the presence of a microorganism that is associated with bacterial vaginosis comprising, contacting a human with one or more of the disclosed fibers wherein the fibers comprise an electrostatically immobilized biological substrate that indicates the presence of one or more biologically active substances produced by a microorganism associated with bacterial vaginosis wherein the indication that a microorganism is present is due to a color change on the one or more fibers.

The disclosed methods relate to a method for determining the presence of a microorganism chosen from Lactobacillus crispatus, Lactobacillus jensenii, Gardnerella vaginalis, Mobiluncus, Bacterocides, and Mycoplasma comprising contacting one or more of the disclosed fibers with a sample and observing a color change on the fiber.

The disclosed methods relate to a method for determining the presence of a microorganism chosen from Lactobacillus crispatus, Lactobacillus jensenii, Gardnerella vaginalis, Mobiluncus, Bacterocides, and Mycoplasma comprising contacting one or more of the disclosed fibers with a sample and observing a odor change associated with the fiber.

### Example 1

Nylon fibers are treated by radiofrequency plasma to form surface reactive amino and carboxyl groups. The fiber is then dipped into a 1% weight/volume solution of poly(glycidyl methacrylate) in methyl ethyl ketone. The fiber is then withdrawn from the graft solution and air dried after which the fiber is placed in a vacuum oven at 105° C. for 10 minutes. Once the substrate anchor is annealed to the fiber the fiber is cooled and analyzed for the thickness of the layer which was found to range from about 5 nm to about 15 nm. The fiber is then dipped in a 1% weight/volume solution of polyethyleneimine in ethanol. The fiber was then air dried after which the fiber is place in a vacuum oven at 100° C. for 1 hour. The fiber is washed with ethanol to remove any unreacted polyethyleneimine.

The surface-modified fiber (˜20 mg) is incubated overnight with 0.5 mL of a 0.5 mg/mL solution of BVBlue™ reagent in 0.5 M potassium acetate buffer (pH 5.5). The fiber is removed and washed with HPLC-grade water then dried in a stream of nitrogen. This fiber is suitable for use in detecting the presence of bacterial vagnosis.

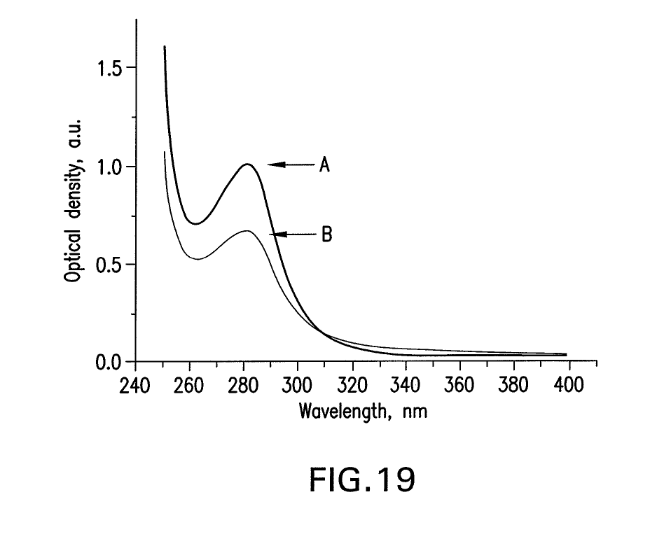

FIG. 19 depicts the UV spectra of A: the initial BVBlue™ absorbance and B: the BVBlue™ absorbance after exposure to the modified fiber. The drop in absorbance indicates reflects the amount of neuraminic acid substrate that is electrostatically immobilized onto the polymer surface. By measuring the change in absorbance at 280 nm the amount of the substrate bound to the fiber is determined to be about 30%.

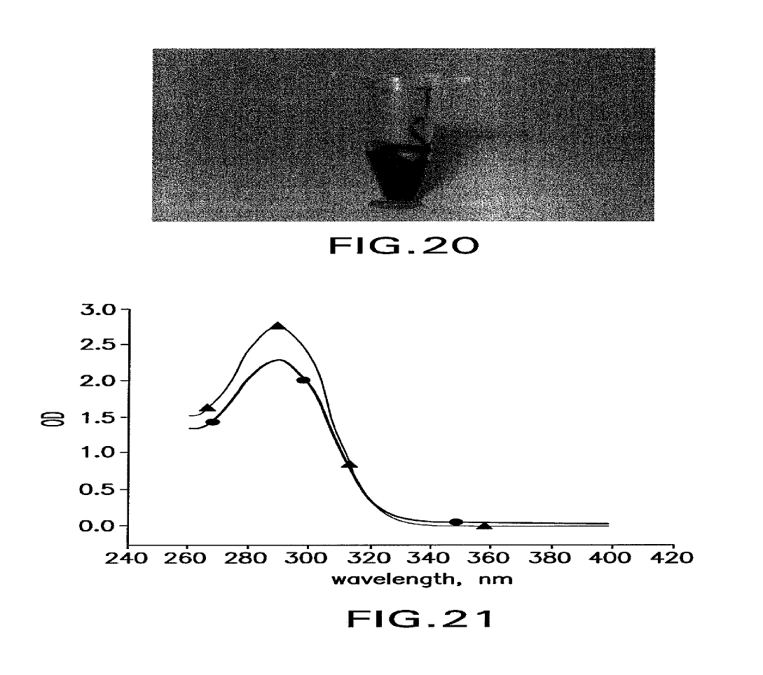

The dried fiber is then tested for activity in the presence of sialidase. Sialidase derived from Arthrobacter ureafeciensi (SigmaAldrich, Cat. No. N8271) is dissolved in 50 mM phosphate buffer at 10 U/mL. The is place into a 0.5 mL test tube and treated with 6 mL of silalidae solution. After 10 minutes of incubation at 37° C., 50 mL of 0.1 M NaOH is added. The solution turns bright blue indicating the release of 5-bromo-4-chloro-1H-indol-3-ol. FIG. 20 depicts a color micrograph of the fiber in solution.

### Example 2

Poly(ethylene-co-terephthalate) fibers are treated by radiofrequency plasma to form surface reactive hydroxyl and carboxyl groups. The fiber is then dipped into a 1% weight/volume solution of poly(glycidyl methacrylate) in methyl ethyl ketone. The fiber is then withdrawn from the graft solution and air dried after which the fiber is placed in a vacuum oven at 105° C. for 10 minutes. Once the substrate anchor is annealed to the fiber the fiber is cooled and analyzed for the thickness of the layer which was found to range from about 5 nm to about 15 nm. The fiber is then dipped in a 1% weight/volume solution of polyethyleneimine in ethanol. The fiber was then air dried after which the fiber is place in a vacuum oven at 100° C. for 1 hour. The fiber is washed with ethanol to remove any unreacted polyethyleneimine.

The surface-modified fiber (˜20 mg) is incubated overnight with 0.5 mL of a 0.5 mg/mL solution of (2R,4S,5R,6R)-5-acetamido-2-(5-bromo-4-chloro-1H-indol-3-yloxy)-4-hydroxy-6-((1R,2R)-1,2,3-trihydroxypropyl)tetrahydro-2H-pyran-2-carboxylic acid in 0.5 M potassium acetate buffer (pH 5.5). The fiber is removed and washed with HPLC-grade water then dried in a stream of nitrogen. This fiber is suitable for use in detecting the presence of bacterial vaginosis.

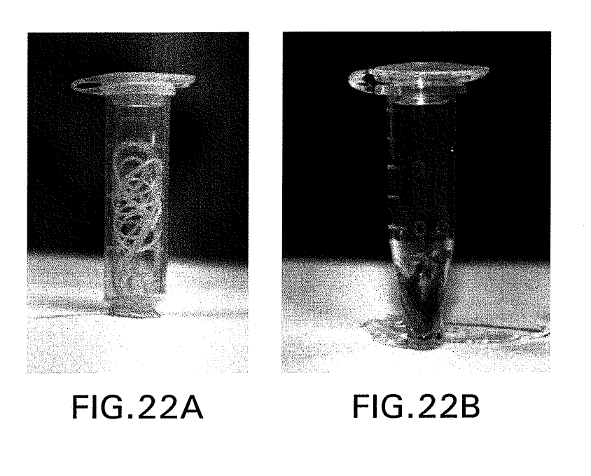

As such, electrostatic attachment of 5-bromo-4-chloro-3-indolyl-α-D-N-acetylneuraminic acid (BCIN) to positively charged nylon fiber also occurs readily upon treatment of PEI-modified fiber with BCIN solution. Spectroscopic studies of the solution before and after incubation with the fiber show ˜18% drop of BCIN concentration (FIG. 21). This drop of concentration corresponds to adsorption of 0.17 μg/cm² (0.23 mg/g) of BCIN. Assuming molecular weight of BUN anion of 536.9, surface density of BCIN coating can be estimated at 1.9 anions/nm², or 53 Å² per BCIN anion, indicative of a monolayer coating. FIG. 6 shows the photograph of the fiber after 2 h incubation with sialidase at 37° C. Notably, use of the fibers treated by BCIN did not require reaction with an alkaline solution to reveal the color, which makes them much more suitable for the proposed applications then those treated by BVBlue reagent. FIG. 21 depicts the UV spectra of 0.58 mg/mL BCIN solution before (▴) and after () its incubation with a positively charged nylon fiber. BCIN-treated nylon fiber changes color from white FIG. 22A to blue FIG. 22B in the presence of 0.06 U of sialidase from Arthrobacter ureafaciens in phosphate buffer saline (pH 5.5).

### Testing Involving Human Biological Samples

Women participating in this test were selected from those presenting to the general gynecology clinic in Greenville, S.C. with a triage diagnosis of vaginal discharge. The population served by this clinic consists mostly of uninsured and Medicaid patients—a group known to be at high risk for BV. After registering with the clinic front office and obtaining informed consent, potential subjects underwent routine vaginal speculum examination, during which a sample of vaginal secretions was obtained using cotton swabs. One swab was treated with a drop of saline solution on a microscope slide for testing using the Amsel criteria according to the standard clinical practice for the institution. A second swab was placed in a sterile microtainer containing a colorimetric test fiber. Clinic personnel recorded whether the test fiber turns blue (positive test) or remains white (negative). Physicians performing the standard Amsel test were blinded to the results of the fiber test. A third cotton swab was smeared on a microscope slide and allowed to air dry for later Gram staining, scoring using the Nugent criteria, and evaluation of bacterial population.

Clinical decisions regarding treatment were based solely on the Amsel testing, which was also used as the gold standard for judging the predictive value of the fiber testing. The ability of each participating physician to properly diagnosis BV was verified prior to study onset using standardized photomicrographs and review of other Amsel criteria.

### Subject Population Information:

Ten patients were recruited for the study. Subjects were females 16-50 years of age. Those of 18 years and older were consenting adults; parental consent was sought for minors <18 years old. All patients were attending the Gynecology Clinic at Greenville Hospital System. A general guideline for subject selection was normal overall health without being treated for any major medical disorder. The following inclusion criteria were used:
Premenopausal
Intact uterus
Non-pregnant
No use of oral or vaginal antibiotics for 7 days prior to presentation
Not menstruating at time of exam
No vaginal hormonal therapy (e.g., Nuvaring™)

Exclusions included use of oral or vaginal antibiotics for 7 days prior to presentation and menstruation at time of exam. Patients receiving vaginal hormonal therapy (e.g., Nuvaring®) within the previous 4 weeks were also excluded from the study. Also excluded were pregnant women, prisoners, institutionalized individuals, or others who may be considered vulnerable populations.

The fibers described in Example 2 were used in this test involving human vaginal fluid. The samples taken from the patients were tested versus Amsel, Wet prep., and Nugent test protocols. A summary of the results of the samples taken from the 10 women with a preliminary diagnosis of bacterial vaginosis are described herein below in Table I.

TABLE I

| Patient No. | Amsel | Wet prep. | Nugent | Fiber Ex. 2 |
|---|---|---|---|---|
| 1 | Pos. | Neg. | 7 | Faint |
| 2 | Pos. | Pos. | 10 | Faint |
| 3 | Pos. | Pos. | 8 | Blue |
| 4 | Pos. | Pos. | 10 | Blue |
| 5 | Neg. | Neg. | 8 | Neg. |
| 6 | Pos. | Pos. | 6 | Neg. |
| 7 | Neg. | Neg. | 6 | Faint |
| 8 | Pos. | Pos. | 5 | Neg. |
| 9 | Pos. | Pos. | 9 | Blue |
| 10 | Neg. | Neg. | 6 | Neg. |

These data indicate a correlation between the Amsel Test, the Nugent Test, and the ability of the disclosed fibers to diagnose the presence of bacterial vaginosis in human samples.

While particular embodiments of the present disclosure have been illustrated and described, it would be obvious to those skilled in the art that various other changes and modifications can be made without departing from the spirit and scope of the disclosure. It is therefore intended to cover in the appended claims all such changes and modifications that are within the scope of this disclosure.

1. A fiber, comprising:
a) a core having a plurality of pores for accepting and retaining a fluid and one or more moieties formed by surface modification of the core that can serve as a substrate anchor for electrostatic immobilization of a signaling substrate; and
b) a signaling substrate.

2. The fiber of claim 1,
wherein the substrate anchor provides one or more sites for electrostatic immobilization of a signaling substrate of an active substance.

3. The fiber of claim 1, further comprising one or more synthetic polymers.

4. The fiber of according to claim 1, further comprising block copolymers having a hydrophilic block and a hydrophobic block, wherein:
a) the hydrophilic block comprises one or more:
i) polyalkylene glycols;
ii) polyvinyl pyrrolidone;
iii) naturally occurring, synthetic, or modified polysacharrides;
iv) peptides;
v) proteins; and
vi) homopolymers or copolymers comprising monomers chosen from ethylene, propylene, isobutylene, 1-butene, 1-hexene, 4-methyl-1-pentene, 1-octene. styrene, vinyl chloride, methacrylate, methylmethacrylate, vinyl alcohol, styrenes, tetrafluoroethylene, vinylbenzocyclobutane, and 1,4-hexadiene; and
b) the hydrophobic block comprises one or more:
i) lactide, glycolide, caprolactone, and mixtures thereof;
ii) polyester, polyhydroxy acids, polyanhydrides, polyorthoesters, polyetheresters, polyesteramides, polyphosphazines, polyphosphoesters, polyphosphates, polyphosphonates, polycarbonates, polyorthocarbonates, polyamides, or copolymers thereof.

5-18. (canceled)

19. The fiber of claim 1, having a surface area of from about 0.01 m²/g to about 5 m²/g.

20-26. (canceled)

27. The fiber of claim 1, having a strength of from about 0.01 cN/dtex to about 8 cN/dtex to about 10 cN/dtex.

28-33. (canceled)

34. The fiber of according claim 1, wherein the fiber core has a coating of from about 1 nm to about 25 nm.

35-39. (canceled)

40. The fiber of claim 1, wherein the fiber is a shaped fiber.

41. (canceled)

42. The fiber of claim 1, wherein the substrate anchor comprises a moiety chosen from —OH, —CO₂H, —CONH₂, —NH₂, —OSO₃H, —SO₃H, —OPO₃H, —PO₃H, and —SH.

43-46. (canceled)

47. The fiber of claim 1, wherein the substrate anchor is a polymeric graft onto the polymer comprising the fiber core.

48. The fiber of claim 1, wherein the fiber further comprises at least one permeable skin.

49-53. (canceled)

54. The fiber of claim 1, wherein the substrate releases a compound having an odor.

55. (canceled)

56. The fiber of claim 1, wherein the signalling substrate has the formula: 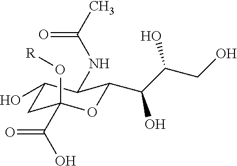
wherein R represents a moiety that when released by reaction of an enzyme with the substrate produces a molecule having the formula ROH.

57-59. (canceled)

60. The fiber of claim 1, comprising a polymer having an average molecular weight of from about 500 daltons to about 2,000,000 daltons.

61-79. (canceled)

80. A method of detecting a biologically active substance comprising contacting a biological fluid or a sample comprising a biological fluid with a fiber of claim 1.

81-85. (canceled)

86. The method of claim 80, wherein the biologically active substance is a microorganism.

87-89. (canceled)

90. The method of claim 86, wherein the microorganism is Lactobacillus crispatus, Lactobacillus jensenii, Gardnerella vaginalis, Mobiluncus, Bacterocides, or Mycoplasma.

91. A fiber, comprising:
a) a core having a plurality of pores for accepting and retaining a fluid wherein the core comprises a surface modified polymer and wherein the core has been modified to comprise a plurality of moieties capable of covalently bonding to a substrate anchor;
b) a first substrate anchor covalently bonded to the core, comprising a polymer, wherein the first substrate anchor is covalently bonded to a second substrate anchor;
c) a second substrate anchor covalently bonded to the first substrate anchor, wherein the second substrate anchor comprises a polymer, wherein the second substrate anchor comprises one or more units that electrostatically immobilize a signaling substrate of an active substance; and
d) a signaling substrate.

92-97. (canceled)

98. The fiber claim 91, wherein the signaling substrate comprises a compound having the formula: 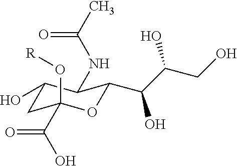
wherein R represents a moiety that when released by reaction of an enzyme with the substrate produces a molecule having the formula ROH.

99-102. (canceled)

103. The fiber of claim 1, wherein at least one of a fiber of claim 1 is incorporated into an article of manufacture.
